(19) United States
(12) Patent Application Publication
(10) Pub. No.: US 2008/0071312 A1
(43) Pub. Date: Mar. 20, 2008

(54) ULTRASONIC TREATMENT OF SKIN CONDITIONS OR DISORDERS, CANCER, AS WELL AS MUSCULAR/SKELETAL REGENERATION

(76) Inventor: Jonathan RIDGWAY, Heslington York (GB)

Correspondence Address: STITES & HARBISON PLLC, 1199 NORTH FAIRFAX STREET, SUITE 900, ALEXANDRIA, VA, 22314, US

(21) Appl. No.: 11/570,828

(22) PCT Filed: Jun. 14, 2005
(86) PCT No.: PCT/GB05/02342

§ 371 Date: Dec. 18, 2006

(30) Foreign Application Priority Data
Jun. 18, 2004 (GB) 0413635.4

(51) Int. Cl. A61N 7/02

(52) U.S. Cl. 607/1; 424/400

(57) ABSTRACT

A method of treating skin and/or skin conditions or disorders including chronoaging by the application of ultrasound.

Skin is subject to deterioration through dermatologic disorders and normal aging (chronoaging) as well as extrinsic factors (environmental). Dermatologic disorders, other than chronoaging include acne, follicular and lesional papules, actinic keratoses, oily skin and rosacea.

Chronoaging results in the thinning and general degradation of skin. As skin naturally ages, there is reduction in the cells and blood vessels that supply the skin. There is also a flattening of the dermal-epidermal junction which results in weaker mechanical resistance. Aging individuals increasingly develop facial fine lines, wrinkles, leatheriness, yellowing or sallowness, sagging, mottling (hyperpigmentation), age spots and the general signs of aging.

Extrinsic factors are primarily those caused by exposure to sun. Changes are most prominent in light skinned individuals who burn easily and tan poorly. The results of photo damage may be identical to those of aging except may appear at an accelerated rate. Wrinkling, yellowing, leatheriness, mottling and hyperpigmentation are all associated with sun damage. Most disturbing to many individuals is the wrinkling effect. As a result, there have been many articles reporting cosmetic treatments aimed at the elimination of wrinkles.

Skin care compositions containing retinoids have become quite prominent in recent years. Retinoic acid, also known as Vitamin A acid or Tretinoin, is well known for treatment of acne. Even more recently, the retinoids have been suggested as treatment against photoaging and sun damage. For instance, U.S. Pat. No. 4,603,146 discloses Vitamin A acid in an emollient vehicle to prevent skin aging. U.S. Pat. No. 4,877,805 suggests a number of retinoids as useful for restoring and reversing sun damage in human skin. EP 0 631 772 describes use of retinal in combination with an irritation ameliorating amount of glycolic acid.

Vitamin A (retinol) is a fat-soluble vitamin found mainly in fish liver oils, liver, egg yolk, butter, and cream. Green leafy and yellow vegetables contain beta-carotene and other provitamin carotenoids which are converted to retinol in cells. Retinol cannot be synthesized in vivo and must be obtained from the diet. Retinol is metabolised into the biologically active derivative retinoic acid (RA) in a variety of cells. The 11-cis isomer of retinol (vitamin A.sub.1 aldehyde), combined with a protein moiety, forms the prosthetic group of photoreceptor pigments in the retina that are involved in night, day and colour vision. Retinol, RA, and other retinoids also influence epithelial cell differentiation.

Retinoids are a large family of molecules encompassing over three thousand members. Retinoic acid, a member of the retinoid family, is a morphogen that defines certain cell fates during development and has the potential to treat cancer by inducing tumor cell differentiation. Retinoid acid binds the retinoic acid receptor (RAR) causing it to form heterodimers with the retinoid X receptor (RXR) and induce gene transcription. In addition to the well accepted role of retinoids in transcription activation, some retinoids may have direct effects on cell second messengers.

Additional therapies that potentiate the toxicity of the immunotoxins to a high degree of specificity for the affected target cell would greatly facilitate treatment of human diseases with these immunotherapeutic agents.

A number of carrier proteins which bind retinol or other retinoids have been identified. These carrier proteins are similar to the fatty acid binding proteins, a family of small (14-16) kDa) cytosolic proteins which bind long-chain fatty acids, fatty acyl-coenzyme A (CoA) derivatives, and other hydrophobic molecules. Two intracellular proteins, cellular retinal-binding protein (CRBP) and cellular retinal-binding protein type II (CRBP II), are found within cells which participate in vitamin A metabolism or function. CRBP, which is expressed in numerous organs and tissues, delivers retinal to specific metabolic enzymes and to specific binding sites within the nucleus, and participates in the transepithelial movement of retinal across blood-organ barriers. CRBP II, which is expressed primarily in the small intestine, appears to be involved in the intestinal absorption of vitamin A.

Retinoid binding proteins appear to direct bound retinoid molecules to specific metabolic pathways. The retinoid binding proteins also protect the cell from the damaging effects of unliganded retinols (such as membrane disruption) and likewise protect structurally unstable retinols from non-enzymatic side reactions (such as isomerization and oxidation). Retinoid binding proteins also appear to function as sensors of retinoid concentration and act as modulators of retinoid metabolism (Napoli, J. L. (1996) FASEB J. 10:993-1001). Retinoid binding proteins have been cloned and characterized from a variety of sources including CRBP from Norway rat and from human (Levin, M. S. et al. (1987) J. Biol. Chem. 262:7118-7124; Colantuoni, V. et al. (1985) Biochem. Biophys. Res. Commun. 130:431-439), and CRBP II from pig and from human (Perozzi, G. et al. (1993) J. Nutr. Biochem. 4:699-705; Loughney, A. D. et al. (1995) Hum. Reprod. 10:1297-1304).

Since retinoids induce differentiation in immature hematopoietic and epithelial cell types, they are potential anti-cancer agents. A promyelocyte cell line was induced to differentiate morphologically and functionally mature granulocytes by incubation with retinoic acid. Other retinoids, including retinal, and retinyl acetate, also induced differentiation, but higher concentrations were required. Retinol is known to affect the differentiation of cultured keratinocytes derived from epidermis and other stratified squamous epithelia. Retinoids and carotenoids have been proposed to have preventative and/or therapeutic effects on lung cancer and cardiovascular disease.

Retinoids have been found to be effective in suppressing tumor development in several carcinogenesis model systems and in human subjects.

It is an object of the present invention to provide a novel method to treat skin.

It is also an object of the present invention to provide a novel method to increase retinoic acid in the cell and/or body.

According to the present invention there is provided a method of enhancing up regulation of the cdx 1 gene by subjecting a cell with ultrasound.

According to the present invention there is also provided a method of enhancing up regulation of the stra 8 gene by subjecting a cell with ultrasound.

Further according to the present invention there is provided a method of enhancing cdx and stra 8 genes by up regulation of the cell with ultrasound.

### Ideally the cell will be a cell of the human body but may be of any animal.

In a further aspect of the present invention there is provided a method of increasing retinoic acid (RA) synthesis in cells by exposing the cells to ultrasound.

Ultrasound has been surprisingly found to increase the synthesis/ metabolisation of retinoic acid (RA) from Retinol in the cell and/or body.

In another embodiment of the present invention there is provided a method of increasing skin radiance without substantial irritation and for treating chronoaging conditions including wrinkles and fine lines, leatheriness, yellowing, sagging, mottling (hyperpigmentation), and age spots and dermatological disorders including dry skin, lightening skin colour, acne, psoriasis, follicular and lesional papules, actinic keratoses, oily skin and rosacea, by treating /exposing the cells, skin and/or body with ultrasound.

In another aspect of the present invention there is provided a method of treating a skin condition selected from the group consisting of dry skin, acne, photo damaged skin, appearance of wrinkles, age spots, increasing stratum corneum flexibility, lightening skin colour, controlling sebum and soothing sensitive skin excretion by treating/exposing the cell with ultrasound.

In a further aspect of the present invention there is provided a method of treating alopecia and leukaemia by treating/exposing the tissue and/or body with ultrasound.

In other aspects of the present invention there is provided a method for stimulating bone growth, healing bone fractures, pseudoarthroses and the like defects in humans and/or animals comprising the step of exposing a cell, tissue and/or body to ultrasound.

There is also provided a method of treating chronoaging conditions such as

Wrinkles, finelines, leatheriness, yellowing, sagging, mottling (hyperpigmentation), age spots, dry skin, lightening skin colour, acne, psoriasis, follicular and leisonal papules, actininc keratoses, oily skin and rosacea

By the step of exposing a cell, tissue e.g. skin, and/or body to ultrasound.

In a further aspect of the present invention there is provided a method for stimulating cartilage growth, healing arthritis and the like defects in humans and/or animals comprising the step of exposing a cell, tissue and/or body to ultrasound.

There is also provided an apparatus for treating bone cartilage and/or skin and any of the above mentioned conditions comprising a means for exposing a cell, tissue or body with ultrasound.

The ultrasound transducer used in the working of the invention may aptly be in contact with the skin or body of the patient transmitting ultrasound pulses to the target site. Suitably the ultrasound will be therapeutic ultrasound of low intensity.

The normal frequency of the ultrasound is 1.5 MHz, the width of each pulse varies between 10 and 2000 microseconds and the pulse repetition rate varies between 100 and 1000 Hz. The power level of the ultrasound is maintained below 100 miliwatts per square centimetre.

Aptly the pulsed radio frequency of the ultrasound would have a frequency in the range of 1.3-2 MHz, and consisting of pulses generated at a rate in the range 100-1000 Hz, with each pulse having a duration in the range 10-2000 microseconds.

However any ultrasound frequency of therapeutic intensity to work the invention would be suitable.

Typical daily treatments may have a duration ideally in the range 1-55 minutes although 10-20 minutes may be preferred. Other duration periods may be used.

There is also provided a method of stimulating retinoic acid cellular response/activity by the step of: exposing a cell, tissue e.g. skin and/or body to ultrasound.

Further, there is provided a method of muscular/skeletal regeneration by the stepf of: exposing a cell, tissue e.g. skin and/or body to ultrasound.

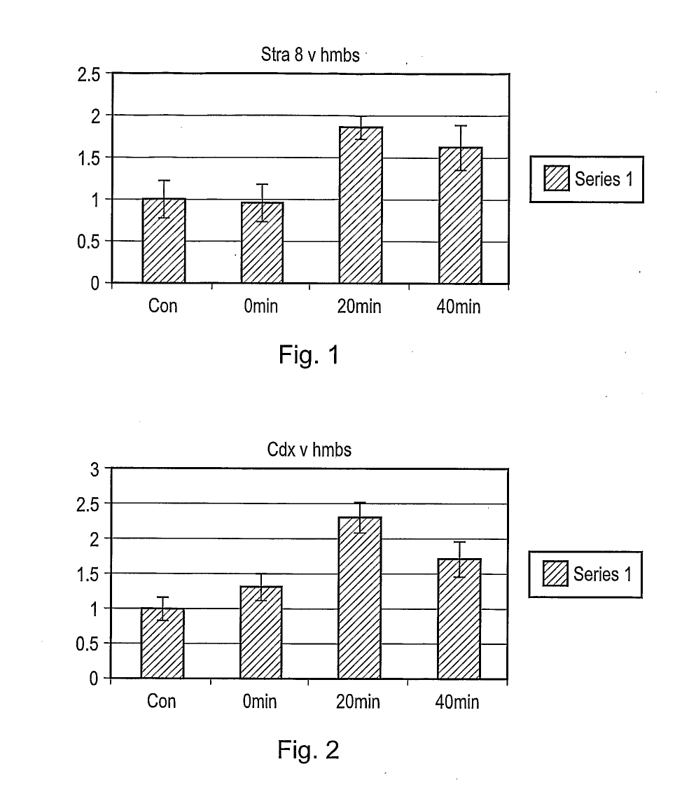

The invention will now be described, by way of example only by the following example, referring to the FIGS. 1 and 2.

FIG. 1 shows the effect of ultrasound on the up regulation of the gene stra 8.

FIG. 2 shows the effect of ultrasound on the up regulation of the gene cdx.

### Method

Cells were exposed to the ultrasound for 20 minutes against controls and the amount/degree of the expression of the genes stra 8 (FIG. 1) and cdx (FIGS. 2) was measured immediately after treatment (0 minute), 20 minutes after treatment (20 minutes) and 40 minutes after treatment (40 minutes). The ultrasound used was provided from a Smith & Nephew Exogen Ultrasound device. The cells used were ST2 cells, mouse stromal cells established from long-term bone marrow culture.

### Results

It was found that upon treating the cells with ultrasound an approximate 2 fold increase in the expression of these two genes was found.

TABLE 1

| | Control | 0 mins | 20 mins | 40 mins | |
|---|---|---|---|---|---|
| Stra 8 | 1 | 0.98002 | 1.833664 | 1.588216 | mean |
| | 0.223664 | 0.251934 | 0.149588 | 0.263762 | std deviation |
| Cdx | 1 | 1.316954 | 2.285988 | 1.705552 | mean |
| | 0.152239 | 0.162431 | 0.230065 | 0.272.539 | std deviation |

### CONCLUSION

The regulation of these two genes are specifically associated with retinoic acid productivity and therefore the ultrasound increases the production of retinoic acid in the cell and body. Increased retinoic acid amounts in the body give the beneficial effects as outlined above.

In another experiment three sets of cells (against controls) were exposed to ultrasound for 20 minutes and their RNA was extracted at 0, 20 and 40 minutes after treatment. The ultrasound was provided by an Exogen device. The cells were ST2 cells, mouse stromal cells established from long-term bone marrow culture.

The RNA was changed to DNA by standard techniques and the resulting DNA was put into a Standard Gene Array (supplied from Super Array) and the resulting responsive indication was measured using a Radiograph detector method of the DNA binding to the genes
CDx1
Stra8
En1
Crbp1
Crbp1
Hoxa1
Stra6

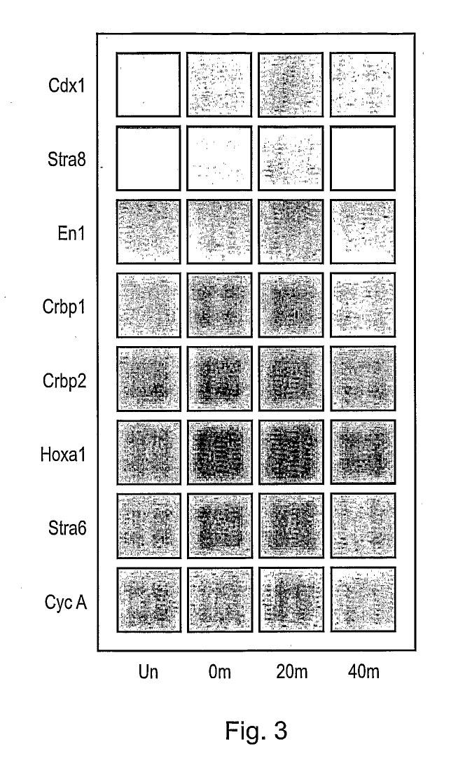

The resulting Radiograph (please see FIG. 3)shows that all the above genes were up regulated by the three sets of cells exposed to ultrasound. The Cyc A gene was a house keeping/control gene.

Therefore 7 out of the 9 genes in the gene pathway of Retinoic Acid were active/upregulated in cells exposed/treated to ultrasound.

This indicates that cells exposed to ultrasound produce retinoic acid and therefore ultrasound can be used to treat and protect skin. The ultrasound may be used to increase skin radiance.

What is claimed is:

1. A method of enhancing the up regulation of the cdx1 gene by subjecting a cell or tissue to ultrasound.

2. A method of enhancing the up regulation of the Stra8 gene by subjecting a cell or tissue to ultrasound.

3. A method of enhancing the up regulation of any one of the genes selected from the group of:
Cdx1, Stra8, En1, Crbp1, Crbp2, Hoxa1, Stra6.

4. A method of increasing retinoic acid synthesis in a cell or tissue by the step of: exposing a cell or tissue to ultrasound.

5. A method of increasing skin radiance by the step of: exposing a cell, tissue e.g. skin and/or body to ultrasound.

6. A method of treating skin by the step of: exposing a cell, tissue e.g. skin and/or body to the ultrasound.

7. A method of treating chronoaging conditions by the step of exposing a cell, tissue e.g. skin and/or body to ultrasound.

8. A method of treating chronaging conditions such as
wrinkles, finelines, leatheriness, yellowing, sagging, mottling (hyperpigmentation), age spots, dry skin, lightening skin colour, acne, psoriasis, follicular and lesconal papules, actinic keratoses, oily skin and rosacea
by the step of: exposing a cell, tissue e.g. skin and/or body to ultrasound.

9. A method of treating dermatological disorders such as dry skin, lightening skin colour, acne, psoriasis, follicular and lesconal papules, actinic keratoses, oily skin and rosacea
by the step of: exposing a cell, tissue e.g. skin and/or body to ultrasound.

10. A method of treating or preventing cancer by the step of: exposing a cell, tissue and/or body to ultrasound.

11. A method of stimulating retinoic acid cellular response/activity by the step of: exposing a cell, tissue e.g. skin and/or body to ultrasound.

12. A method of muscular/skeletal regeneration by the step of: exposing a cell, tissue e.g. skin and/or body to ultrasound.
